(19) United States
(12) Patent Application Publication
(10) Pub. No.: US 2007/0039085 A1
(43) Pub. Date: Feb. 22, 2007

(54) ADJUSTABLY FITTED PROTECTIVE APPAREL WITH ROTARY TENSION ADJUSTER

(75) Inventors: Ian D. KOVACEVICH, Charlotte, NC (US); Kevin J. DAHLQUIST, Charlotte, NC (US); Tom PHILPOTT, Charlotte, NC (US); Daniel Lee BIZZELL, Davidson, NC (US)

Correspondence Address: TILLMAN WRIGHT, PLLC, PO BOX 471581, CHARLOTTE, NC, 28247, US

(73) Assignee: ENVENTYS, LLC, Charlotte, NC (US)

(21) Appl. No.: 11/382,706

(22) Filed: May 11, 2006

Related U.S. Application Data
(63) Continuation of application No. 11/276,400, filed on Feb. 27, 2006.
(63) Continuation-in-part of application No. 11/276,357, filed on Feb. 24, 2006.
Application No. 11/276,357 is a continuation-in-part of application No. 11/222,549, filed on Sep. 9, 2005, now abandoned.
Application No. 11/222,549 is a continuation-in-part of application No. 11/123,942, filed on May 6, 2005.
(60) Provisional application No. 60/656,335, filed on Feb. 25, 2005.
(60) Provisional application No. 60/569,304, filed on May 7, 2004.
(60) Provisional application No. 60/608,397, filed on Sep. 9, 2004.
(60) Provisional application No. 60/656,335, filed on Feb. 25, 2005.

(51) Int. Cl. A42B 1/04; A61F 9/02

(52) U.S. Cl. 2/205; 2/427

(57) ABSTRACT

An article of adjustably fitted protective apparel includes a vest and an adjustable tensioning system for selective tensioning of the vest into a snug fit on a body of a wearer of the vest. The adjustable tensioning system includes a rotary tension adjuster disposed on the vest and including a housing and a first spool disposed within the housing, and a tensioning line extends from the housing. The tensioning line is attached directly or indirectly to the vest. A portion of the tensioning line is configured to be wound upon the first spool during manipulation of the rotary tension adjuster in a first rotational direction thereof. Winding of the portion of the first tensioning line draws together first portions of the vest for providing a snug fit of the first portions.

### I. CROSS-REFERENCE TO RELATED APPLICATIONS

This application is a continuation of, and claims priority under 35 U.S.C. § 120 to, U.S. patent application Ser. No. 11/276,400, filed Feb. 27, 2006, which published as U.S. Patent Application Publication No. US 2006/______ , which is a nonprovisional of, and claims priority under 35 U.S.C. § 119(e) to, U.S. Provisional Patent Application No. 60/656,335, filed Feb. 25, 2005, and which is a continuation-in-part of: (1) U.S. patent application Ser. No. 11/276,357, filed Feb. 24, 2006, which published as U.S. Patent Application Publication No. US 2006/______, and which is a continuation-in-part of each of (A) U.S. patent application Ser. No. 11/222,549, filed Sep. 9, 2005, which is a continuation-in-part of U.S. patent application Ser. No. 11/123,942, filed May 6, 2005, which published as U.S. Patent Application Publication No. US 2006/0015988 A1, and which is a nonprovisional of 60/569,304, 60/608,397, and 60/656,335, and (B) U.S. patent application Ser. No. 11/123,900 filed May 6, 2005, which published as U.S. Patent Application Publication No. US 2005/0247813 A1, and which is a nonprovisional of 60/569,304, 60/608,397, and 60/656,335; (2) U.S. patent application Ser. No. 11/123,942, filed May 6, 2005, which published as U.S. Patent Application Publication No. US 2006/0015988 A1, and which is a nonprovisional of 60/569,304, 60/608,397, and 60/656,335; and (3) U.S. patent application Ser. No. 11/123,900, filed May 6, 2005, which published as U.S. Patent Application Publication No. US 2005/0247813 A1, and which is a nonprovisional of 60/569,304, 60/608,397, and 60/656,335. The entire disclosure of each of these patent applications and patent application publications is hereby incorporated herein by reference.

### II. COPYRIGHT STATEMENT

All of the material in this patent document is subject to copyright protection under the copyright laws of the United States and of other countries. The copyright owner has no objection to the facsimile reproduction by anyone of the patent document or the patent disclosure, as it appears in the Patent and Trademark Office patent file or records, but otherwise reserves all copyright rights whatsoever.

### III. BACKGROUND OF THE PRESENT INVENTION

“Body armor” generally is understood to encompass an article of protective clothing that provides a measure of protection against projectiles fired from handguns, shotguns and rifles. Body armor is typically worn by members of police forces, the military personnel, and private security personnel. Body armor commonly is referred to as a bulletproof vest; however, this may be a misnomer, as the vests may provide little or no protection against rifle ammunition or even against handgun ammunition fired from a pistol-caliber carbine, depending upon the level of armor of the vest. As used herein, the term “vest” is intended to refer to such body armor. A generic example of such a vest is illustrated in FIG. 1.

Vests commonly are made from Kevlar™ material. Vests further may be augmented with metal, such as steel or titanium; ceramic; or polyethylene plates that provide additional protection to vital areas above and beyond protection provided by Kevlar™ material. Known as “trauma plates,” this additional armor plating has proven effective against bullets fired from handguns and some rifles. Such vests have become standard in military use, as advances in ballistic technology have rendered vests made only from Kevlar™ materials generally ineffective. Titanium backing typically is used.

In principal, a vest does not protect the wearer by deflecting a bullet. Instead, the individual layers of material catch the bullet and spread its force over a larger portion of the body, deforming the round and stopping the bullet before it can penetrate into the body. While a vest can prevent a bullet from penetrating, the wearer could still receive blunt force trauma from the momentum of the bullet, with results ranging from bruises to serious internal injuries. Also, it offers little protection against bladed weapons, such as knives or arrows, because these weapons rely on penetration. Bullets, on the other hand, depend on sharp impact, which is what the vest is designed to counter. To protect against the latter form of attack, an outer surface of a vest may be coated with tiny crystals of a sandpaper-like material or placing a very thin plate of resin hardened glass-fiber sheet between the Kevlar™ layers.

When worn, it is important for a vest to be disposed in snug fit with the body of the wearer. The protection afforded by the vest may be degraded if the vest is worn in a loose fit permitting play between the vest and the body. On the other hand, a snug fit may be less comfortable than a loose fit, and a wearer may prefer the ability to alternate between a loose fit and a snug fit depending on the currently perceived threat level.

A vest conventionally is secured onto the body of wearer in a snug fit using straps having releasable fasteners. Such releasable fasteners include hook-and-loop engagement elements, such as Velcro™ fasteners, and/or quick-release buckles. Furthermore, the straps themselves may be resilient. Dragone et al. U.S. Pat. No. 5,373,582; Davis et al. U.S. Pat. No. 5,797,140; and Bachner, Jr. U.S. Pat. No. 6,047,399 collectively disclose such representative straps and fasteners for vests, the entirety of each patent of which is incorporated herein by reference.

Unfortunately, it can be cumbersome to loosen a vest into a loose fit disposition, and/or to tighten a vest into a snug fit disposition, for several reasons. First, loosening or tightening the vest generally requires manipulation of two or more fasteners. For example, the loosening of each side portion of a vest generally requires the release of at least one fastener corresponding to each side portion. The further loosening of the shoulders of the vest similarly generally requires the release of at least one fastener corresponding to each shoulder, if the vest can be loosened in the shoulders at all. Second, access to the required fasteners may be inhibited, such as by clothing if the vest is worn under one or more garments. Access to the required fasteners also may require access to different areas of the vest, including a right hand side of the vest and a left hand side of the vest. Moreover, such access may require the use of each hand as opposed to use of either just the left or the right hand.

Accordingly, in view of the foregoing, it has been recognized that a need exists for improvements in vests relating to the selective tensioning of a vest and the selective loosening of a vest. One or more such needs are met by one or more vests in accordance with embodiments of the present invention.

### IV. SUMMARY OF THE PRESENT INVENTION

The present invention includes many aspects and features. In one aspect of the invention, an article of adjustably fitted protective apparel includes a vest and an adjustable tensioning system for selective tensioning of the vest.

In accordance with this aspect, the adjustable tensioning system includes a rotary tension adjuster that is disposed on the vest. The rotary tension adjuster includes a housing and a first spool disposed within the housing. A first tensioning line extends from the housing and is attached to the vest. A portion of the first tensioning line is configured to be wound upon the first spool during manipulation of the rotary tension adjuster in a first rotational direction thereof, and winding of the portion of the first tensioning line onto the first spool results in drawing of first portions of the vest together for providing a snug fit of the first portions of the vest when worn.

The attachment of the tensioning line to the vest may be direct or indirect. For instance, the first tensioning line may be attached indirectly to the vest by a quick-release buckle that is attached to the vest.

The vest may comprise a ballistic vest having one or more protective panels. The first tensioning line is flexible and capable of conveying tension. Furthermore, the first tensioning line may comprise a mono-filament material or a multi multi-filament material. The first tensioning line further may be a cable, and may be made from nylon.

In a feature, the first tensioning line extends from the rotary tension adjuster and also returns to the rotary tension adjuster.

In another feature, the adjustable tensioning system further includes a second spool disposed within the housing and a second tensioning line extending from the housing and attached directly or indirectly to the vest. A portion of the second tensioning line is configured to be wound upon the second spool during manipulation of the rotary tension adjuster in a second rotational direction thereof, and winding of the portion of the second tensioning line on the second spool results in the drawing together of second portions of the vest for providing a snug fit of the second portions of the vest when worn.

With further regard to this feature, a third tensioning line also may extend from the housing and be attached directly or indirectly to the vest, and a portion of the third tensioning line also may be configured to be wound upon the first spool during manipulation of the rotary tension adjuster in the first rotational direction thereof, whereby winding of the portion of the third tensioning line on the first spool results in the drawing together of third portions of the vest for providing a snug fit of the third portions of the vest when worn.

Moreover, a fourth tensioning line also may extend from the housing and be attached directly or indirectly to the vest, and a portion of the fourth tensioning line may be configured to be wound upon the second spool during manipulation of the rotary tension adjuster in the second rotational direction thereof, whereby winding of the portion of the fourth tensioning line on the first spool results in the drawing of fourth portions of the vest together for providing a snug fit of the fourth portions of the vest when worn.

Additionally, the first and third tensioning lines may form a continuous loop and the second and fourth tensioning lines may form a continuous loop. The first portions of the vest may comprise front and rear shoulder portions of the vest, the third portions of the vest may comprise front and rear shoulder portions of the vest, the second portions of the vest may comprise front and rear side portions of the vest, and the fourth portions of the vest may comprise front and rear side portions of the vest. The first and third tensioning lines may be attached to the shoulder portions of the vest, and the second and third tensioning lines may be attached to the side portions of the vest.

In another feature of this aspect, the first tensioning line is attached to a clip that is releasably fastened to an anchor location out of a plurality of anchor locations that are located on the vest. The plurality of anchor locations may be disposed along a portion of the vest at increasing distances from the rotary tension adjuster. These anchor locations further may be defined by a band that is attached to the vest, and the band may be formed from an elastic material and include elastic characteristics.

In still yet another feature of this aspect, the adjustable tensioning system further comprises a second rotary tension adjuster disposed on the vest from which a second tensioning line extends. The second tensioning line is attached directly or indirectly to the vest, and a portion of the second tensioning line is configured to be wound upon a first spool of the second rotary tension adjuster during manipulation of the second rotary tension adjuster in a first rotational direction thereof, whereby the winding of the portion of the second tensioning line on the second spool results in the drawing of second portions of the vest together for providing a snug fit of the second portions of the vest when worn.

In another feature of this aspect, further manipulation of the rotary tension adjuster permits separation of the first portions of the vest, thereby providing a loose fit of the first portions of the vest when worn.

In still yet another feature of this aspect, the rotary tension adjuster is generally centered on a front portion of the vest.

In another feature, the rotary tension adjuster is disposed within a pocket of the vest. The pocket includes an opening through with a dial of the rotary tension adjuster extends for manipulation thereof by a wearer of the vest.

In still yet another feature of this aspect, the adjustable tensioning system further includes a distribution channel system through which the tensioning line extends. A first distribution channel of the distribution channel system sheathes the first tensioning line.

With further regard to this feature, the first distribution channel may comprise an elongate section that sheathes the first tensioning line and an outrigger section that extends from the elongate section to provide stability and support to the elongate section during tensioning of the first tensioning line. The first distribution channel also may comprise a layer of material that is attached to a surface of the vest in overlapping relation to the first tensioning line and include a tubular member through which the first tensioning line extends, with the layer of material overlapping the tubular member in which the first tensioning line extends. The distribution channel system further may includes C-shaped sections proximate side portions of the vest and/or proximate shoulder portions of the vest.

In another feature of this aspect, the first tensioning line fully encircles a wearer of the vest by extending from the rotary tension adjuster, passing through a left upper torso distribution channel formed in a front portion of the vest, passing through a left line spreader, passing through a distribution channel formed in a back portion of the vest, passing through a right line spreader, passing through a right upper torso distribution channel formed in another front portion of the vest, and returning to the rotary tension adjuster.

In still yet another feature, a second tensioning line extends from the housing and is attached directly or indirectly to the vest. A portion of the second tensioning line is configured to be wound upon the first spool during manipulation of the rotary tension adjuster in the first rotational direction thereof, whereby winding of the portion of the second tensioning line on the first spool also results in the drawing of the first portions of the vest together for providing a snug fit of the first portions of the vest when worn.

With further regard to this feature, the first tensioning line also includes an upper side torso tensioning line that extends from the rotary tension adjuster toward a side portion of the vest, and wherein the second tensioning line comprises a lower side torso tensioning line that extends from the rotary tension adjuster toward the side portion below and in spaced relation to the upper side torso tensioning line.

Furthermore, both the upper side torso tensioning line and the lower side torso tensioning line may be attached to the side portion of the vest by a line spreader. The line spreader may be attached to a back portion of the vest by a quick-release buckle. The line spreader also may comprises a rigid, elongate member that maintains a fixed distance between opposed ends thereof, and the upper and lower side torso tensioning lines may be attached to the line spreader at respective opposed ends thereof, whereby bunching or gathering of the vest about the midriff is prevented during tensioning of the side portions.

The upper and lower side torso tensioning lines also may comprise separate lines that are attached at opposite respective ends thereof to the line spreader and to the rotary tensioner adjuster; and the upper and lower torso lines may pass through the line spreader and through distribution channels formed in the side portions of the vest to attach to a back portion of the vest. Alternatively, the upper and lower side torso tensioning lines are respective portions of a single line; the single line forming the upper and lower side torso tensioning lines may slide freely through a channel defined by the line spreader; and an end of the single line may be anchored to the rotary tension adjuster with the opposite end being spooled within the rotary tension adjuster or opposite ends of the single line both may be spooled within the rotary tension adjuster.

In still yet another feature, the rotary tension adjuster is selectively detachable and reattachable to a plurality of different locations on a front portion of the vest. In this regard, the adjustable tensioning system may include a base plate upon which the rotary tension adjuster is mounted, the base plate may include hook-and-loop fasteners, and the front portion of the vest may include hook-and-loop fasteners that couple with the hook-and-look fasteners of the base place for selective detachment and reattachment of the base plate to the front portion of the vest at different locations.

In another feature, the adjustable tensioning system includes a joiner connecting the first tensioning line to a plurality of tensioning belts, each tensioning belt being attached to the vest. The joiner may link the first tensioning line to three tensioning belts, and the first tensioning line may be attached to the joiner for rotational movement of the joiner relative to the first tensioning line at the location of the attachment.

In still another feature, the first tensioning line extends from the rotary tension adjuster to a side portion of the vest and is connected to an elastic cord that passes freely through loops respectively attached to the front and rear side portions of the vest, thereby defining serpentine configuration of the elastic cord. The loops may be disposed in fixed spaced relation by a rigid spreader bar for preventing bunching, gathering or migration of the rear side portions of the vest during tensioning of the first tensioning line.

In another aspect of the invention, an article of adjustably fitted protective apparel includes a vest and a rotary tension adjuster that draws within a housing of the rotary tension adjuster a tensioning line during manipulation of the rotary tension adjuster in a first rotational direction thereof. The apparel further includes means for tensioning a portion of the vest into a snug fit on a body of a wearer of the vest as a result of the drawing, into the housing of the rotary tension adjuster, of the tensioning line during manipulation of the rotary tension adjuster in the first rotational direction thereof.

In a feature of this aspect, the rotary tension adjuster further draws in to the housing thereof another tensioning line during manipulation of the rotary tension adjuster in a second rotational direction thereof; and the apparel further includes means for tensioning the vest into a snug fit on a body of a wearer of the vest as a result of the drawing, into the housing of the rotary tension adjuster, of the other tensioning line during manipulation of the rotary tension adjuster in the second rotational direction thereof.

In yet another aspect of the invention, a method for adjustably tensioning an article of protective apparel includes the step of manipulating a rotary tension adjuster in a first rotational direction thereof to draw together first sections of a vest.

In a feature of this aspect, the method further includes the step of manipulating the rotary tension adjuster in a second rotational direction thereof opposite to the first rotational direction thereof to draw together second sections of the vest. Each step of manipulating the rotary tension adjuster preferably comprises manually rotating a dial of the rotary tension adjuster.

In addition to the aforementioned aspects and features of the present invention, it should be noted that the present invention further includes the various possible combinations of such aspects and features.

Further applicability of adjustable tensioning systems in accordance with embodiments of the present invention will become apparent from the detailed description provided hereinafter. It should be understood that the detailed description and specific examples, while setting forth one more preferred embodiments of the invention, are intended for purposes of illustration only and are not intended to limit the scope of the invention.

### V. BRIEF DESCRIPTION OF THE DRAWINGS

Further aspects, features, embodiments, and advantages of the present invention will become apparent from the following detailed description with reference to the drawings, wherein:

FIG. 1 is a generic example of a vest;

FIG. 2A is a front view of an article of adjustably fitted protective apparel, worn by a user, according to a first embodiment of the present invention;

FIG. 2B is a front view of the article of protective apparel of FIG. 2A, still worn by a user, but shown with an outer cover removed for clarity;

FIG. 2C is a front view of the article of protective apparel of FIG. 2A, shown in an unworn disposition;

FIG. 3A is a side cross-sectional view of a portion of the right side of the article of protective apparel of FIG. 2C, taken along line 3-3;

FIG. 3B is a side cross-sectional view of a portion of the right side of a variation of the article of protective apparel 10 of FIGS. 2A-C;

FIG. 3C is a side cross-sectional view of a portion of the right side of another variation of the article of protective apparel 10 of FIGS. 2A-C;

FIGS. 4A-4D are front views of an article of adjustably fitted protective apparel according to a second embodiment of the present invention;

FIGS. 5A and 5B are front views of an article of adjustably fitted protective apparel according to a third embodiment of the present invention;

FIG. 6 is a front view of the article of adjustably fitted protective apparel of FIG. 5A;

FIG. 7 is a front view of an article of adjustably fitted protective apparel 310 according to a fourth embodiment of the present invention;

FIG. 8 is a front view of an article of adjustably fitted protective apparel 410 according to a fifth embodiment of the present invention;

FIG. 9 is a front view of an article of adjustably fitted protective apparel 510 according to a sixth embodiment of the present invention; and

FIG. 10 is a front view of an article of adjustably fitted protective apparel 610 according to a seventh embodiment of the present invention.

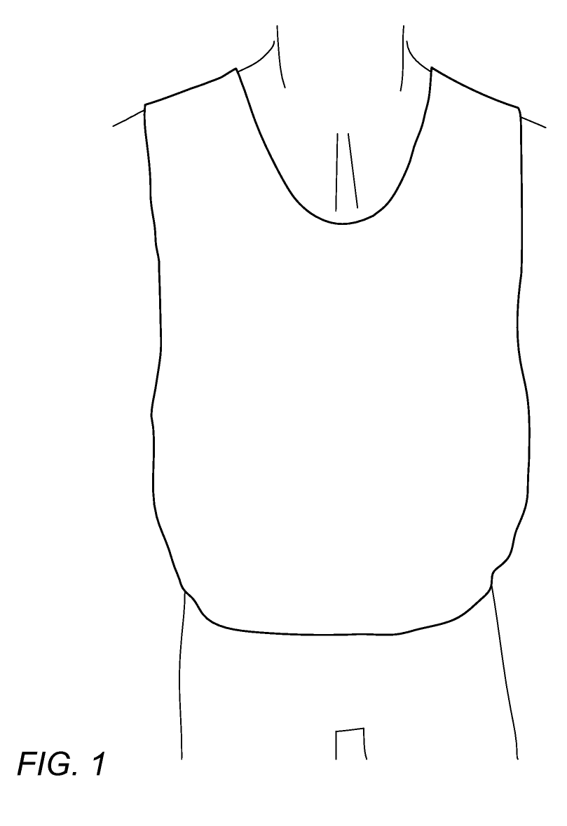

### VI. DETAILED DESCRIPTION OF THE PREFERRED EMBODIMENTS

As a preliminary matter, it will readily be understood by one having ordinary skill in the relevant art (“Ordinary Artisan”) that the present invention has broad utility and application. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Referring now to the drawings, in which like numerals represent like components throughout the several views, protective apparel having adjustable tensioning systems in accordance preferred embodiments of the present invention are next described. The following description of preferred embodiments is merely exemplary in nature and is in no way intended to limit the invention, its application, or uses.

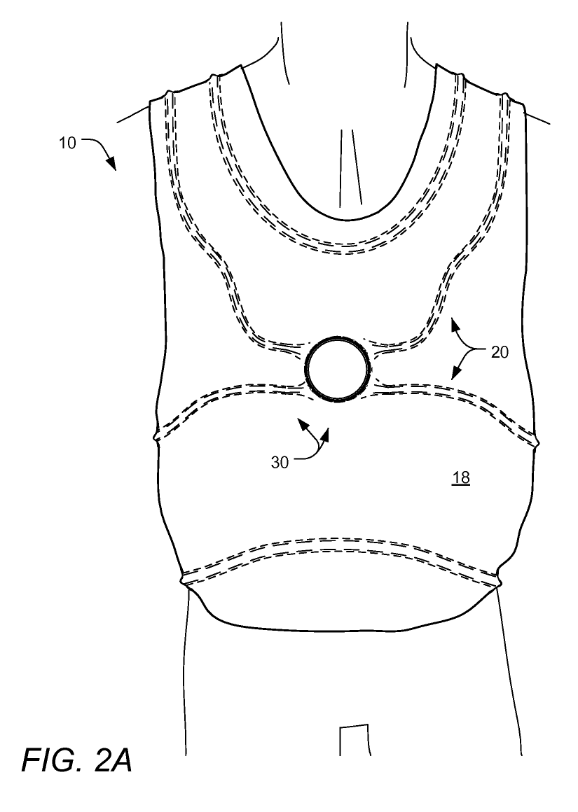

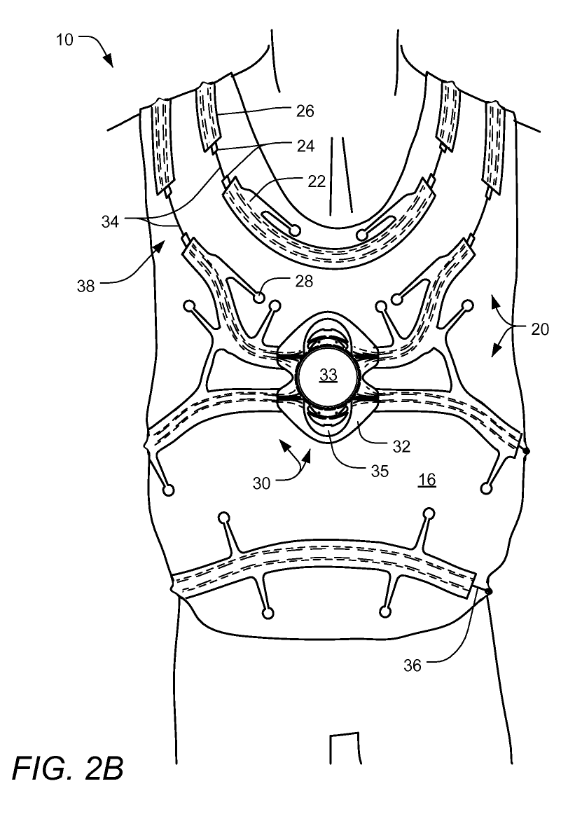

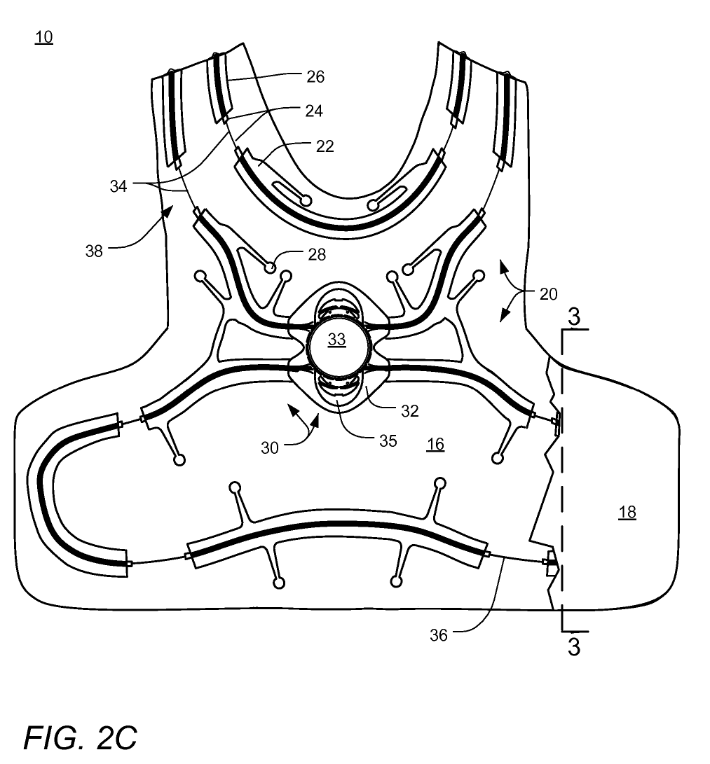

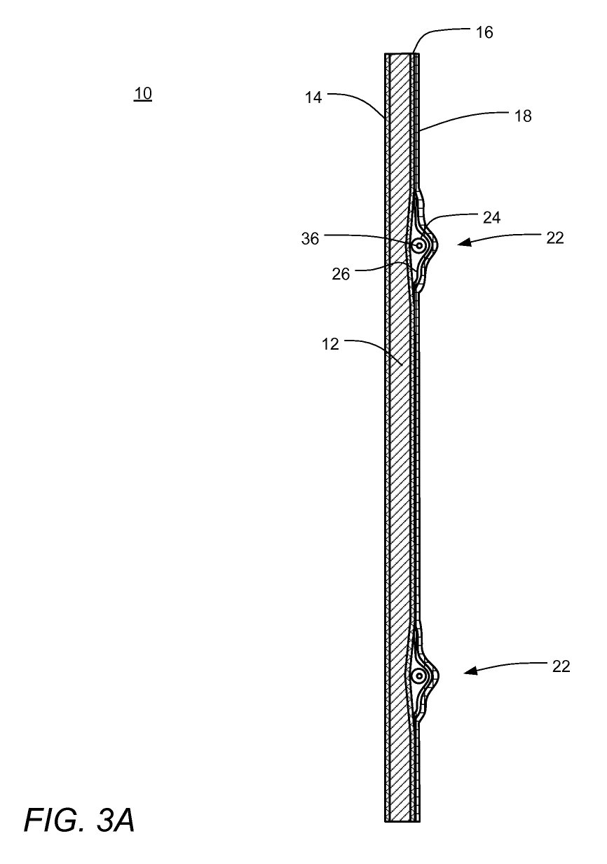

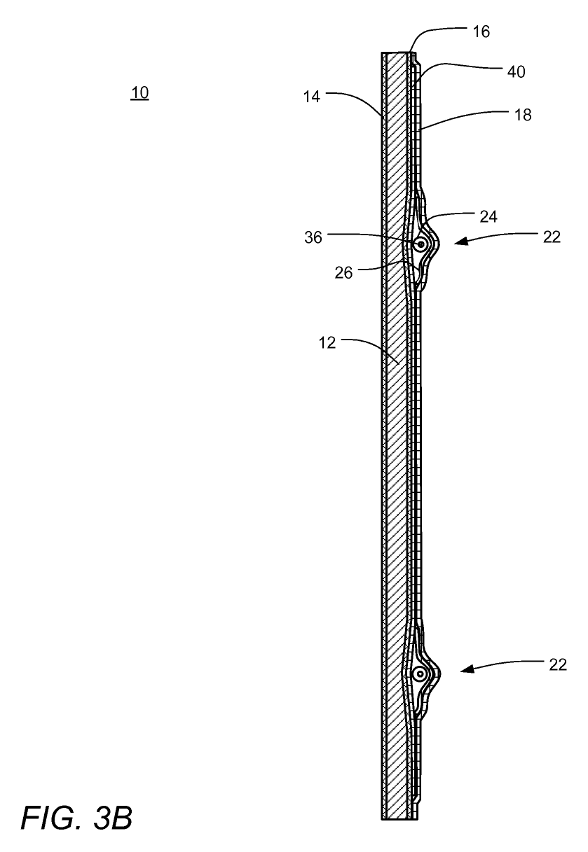

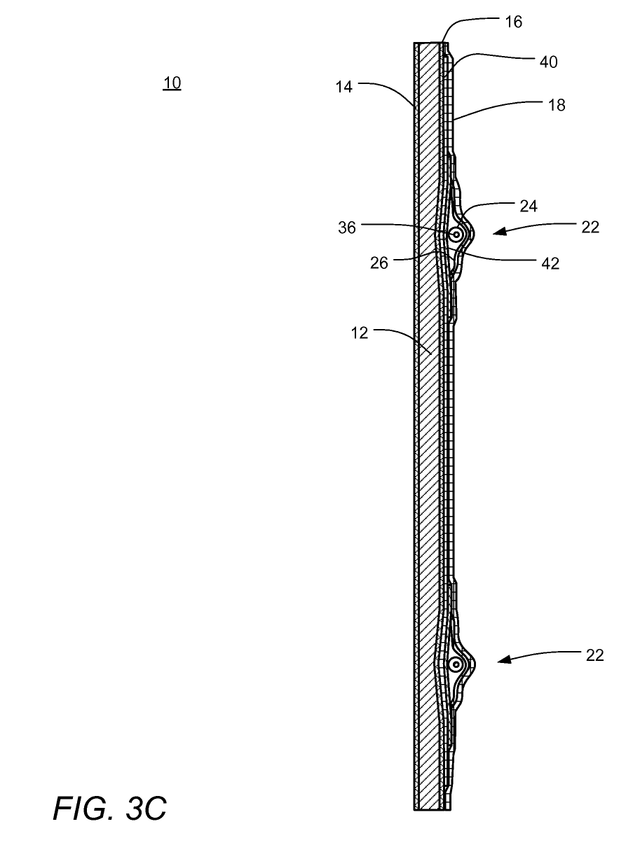

FIG. 2A is a front view of an article of adjustably fitted protective apparel 10, worn by a user, according to a first embodiment of the present invention; FIG. 2B is a front view of the article of protective apparel 10 of FIG. 2A, still worn by a user, but shown with an outer cover 18 removed for clarity; FIG. 2C is a front view of the article of protective apparel 10 of FIG. 2A, shown in an unworn disposition; and FIG. 3A is a side cross-sectional view of a portion of the right side of the article of protective apparel 10 of FIG. 2C, taken along line 3-3. As collectively shown therein, the illustrated article 10, often generally referred to hereinafter as a bullet-proof vest, comprises one or more protective panels 12, an inner cover 14, a middle cover 16, an outer cover 18, a channel distribution system 20 and an adjustable tensioning system 30.

The adjustable tensioning system 30 comprises a rotary tension adjuster 32 and a plurality of tensioning lines 34, 36, while the channel distribution system 20 includes a plurality of distribution channels 22. As illustrated, the end portions of each tensioning line 34, 36 are spooled or otherwise gathered within the rotary tension adjuster 32. Each tensioning line 34, 36 extends in a continuous loop from the rotary tension adjuster 32 proximal the sternum area of the vest 10, passes through various distribution channels 22 formed or disposed in the front portion of the vest 10, finally returning again to the rotary tension adjuster 32. Manipulation of the rotary tension adjuster 32 in one sense effects the withdrawing of portions of the tensioning lines from the distribution channels 22 into the adjuster 32, thereby tightening of the vest 10. Manipulation of the rotary tension adjuster 32 in another sense effects the releasing of portions of the tensioning lines 34, 36 from the adjuster 32, thereby loosening the vest 10.

As shown in FIG. 3A, the channels 22 of the channel distribution system 20 preferably include a tubular member 24 disposed between a layer of material or sheath 26 and the middle cover 16. The tensioning lines 34, 36 are routed through the tubular members 24, thereby minimizing friction and snagging within the body of the vest 10. The tubular members may be made of a suitable flexible plastic material with relatively low frictional characteristics and of a diameter to permit the tensioning lines to be guided smoothly therethrough. The sheaths 26 may be made of nylon or other suitable material and may be fastened to the middle cover 16 using any appropriate means, including sewed seams, hook-and-loop fasteners, gluing or cementing, or the like. Each tensioning line 34, 36 is generally strong and capable of supporting tension, is flexible and is capable of being wound about a spool. Tensioning lines 34, 36 each may comprise a mono-filament material or a multi-filament material and may be natural or synthetic fibers. Alternately the tensioning lines 34, 36 may be formed of one or more metals and, for example, may comprise cables. Alternately the tensioning lines 34, 36 may be formed from nylon.

Each distribution channel further may comprise an elongate section, which sheathes the a tensioning line, as well as an outrigger section 28, which extends from the elongate section to provide stability and support to the elongate section during tensioning of the lines.

Returning to FIGS. 2B and 2C, wherein the outer cover 18 has been removed for clarity, gaps 38 in the distribution channels 22 may be readily observed. The gaps 38, which define separate sections of both the tubular members 24 and the sheath 26, allow the different sections of the distribution channels 22 to move toward or away from each other, as appropriate, when the tensioning lines 34, 36 are being tightened or loosened, respectively. In other words, when a tensioning line 34, 36 is tightened, one tubular member section 24 is allowed to move toward another section 24, and the corresponding sheath sections 26 are likewise allowed to move toward each other.

Optionally, yoke portions of the vest 10 that extend from the pectoral area, passing over the collar bone area, and toward the shoulder blade area are constructed of extensible fabric that is elasticized to stretch and retract to avoid bunching or gathering of the material as the fit of the vest 10 is adjusted and as the wearer of the vest 10 moves about. Similarly, side portions of the vest 10 that extend from the frontal torso to the rear torso passing over the ribs under the arms are optionally constructed of extensible fabric. Thus, while protective front and back portions of the vest 10 may each comprise woven carbon fiber ballistic shield material that is not generally extensible, an adjustable and comfortable fit is facilitated by the rotary tension adjuster 32, multiple tensioning lines 34, 36, and extensible yoke and side portions of the vest 10.

The distribution channels 22 formed in the front portion of the vest 10 are made more apparent in FIG. 2C by the visibility of the tensioning lines 34, 36. As illustrated in FIGS. 2B and 2C, a first tensioning line 34 is routed from the rotary tension adjuster 32 up over a first shoulder of the wearer toward the upper back section of the vest 10, then is routed back forward over the same shoulder, down toward the upper chest, then back up to the second shoulder of the wearer and again toward the upper back section of the vest 10, then is routed back forward over the second shoulder and back to the rotary tension adjuster 32. A second tensioning line 36 is routed from the rotary tension adjuster 32 toward an upper portion of a first side of the wearer's torso, around toward the lower back of the wearer, then back around a lower portion of the same side of the wearer's torso, across the lower stomach region of the wearer to a lower portion of the second side of the wearer's torso, and finally back around an upper portion of the second side of the wearer's torso and back to the rotary tension adjuster 32. However, it will be apparent that other routings may be utilized, that other numbers of tensioning lines 34, 36 may be utilized, and that other alternative features may be employed, such as those described hereinbelow with regard to this and other embodiments of the present invention, all without departing from the scope of the present invention.

FIG. 3B is a side cross-sectional view of a portion of the right side of a variation of the article of protective apparel 10 of FIGS. 2A-C. In this variation of the article of protective apparel 10 of FIGS. 2A-2C, a first hook-and-loop layer 40 is sandwiched between the middle cover 16 and the outer cover 18, and the distribution channels are attached to the outer surface of the first hook-and-loop layer 40. This may permit a user to selectively adjust the location of the various distribution channel sections 22, each independently from the others. Still greater selectivity may be provided by the additional variation of the article of protective apparel 10 as shown in FIG. 3C. In this variation, the distribution channel sections 22 may be attached to additional hook-and-loop panels 42 which in turn may be selectively located on the first hook-and-loop layer 40. Access to the distribution channels 22 and the hook-and-loop materials may be facilitated by providing a removable outer cover 18 or by providing access openings and the like (not shown in FIGS. 2A-2C, but suitable examples of which are shown with regard to other embodiments of the present invention, described below). Alternatively, the distribution channels may be welded or otherwise permanently adhered to a surface of a layer of the vest in a predefined layout on the surface.

The rotary tension adjuster 32 of a preferred embodiment of the invention comprises one of the coplanar cranks disclosed in U.S. patent application Ser. No. 11/276,357. Other rotary tension adjusters or cranks 132 also may be used, including those of the other incorporated references, such as the coaxial cranks disclosed in U.S. patent application Ser. No. 10/129,900.

Although a complete description of such a dual-spool rotary tension adjuster 32 is provided in the referenced patent applications and thus is not provided herein, the rotary tension adjuster 32 may be generally described as follows. Two spools within the rotary tension adjuster 32 are driven to coil and tension the respective lines 34, 36 by rotation of a disk-shaped handle or dial 33 that is manually turned. Manual turning of the dial 33 in a first rotational direction, for example clockwise, drives (by geared engagement) a first spool to thereby coil and tension one or more lines 34. Manual turning of the dial 33 in a second rotational direction opposite the first rotational direction, for example counter-clockwise, drives a second spool to thereby coil and tension one or more other lines 36. A pair of symmetric opposed end portions 35 of the rotary tension adjuster 32 are provided for releasing the spools to rotate freely. Each end portion 35 corresponds to and releases a particular one of the two spools of the rotary tension adjuster 32. The pressing by the user of one of the end portions 35 releases a respective spool to rotate freely, thereby releasing the tension in the respective line 34, 36 and allowing extension of the line 34, 36 from the rotary tension adjuster 32.

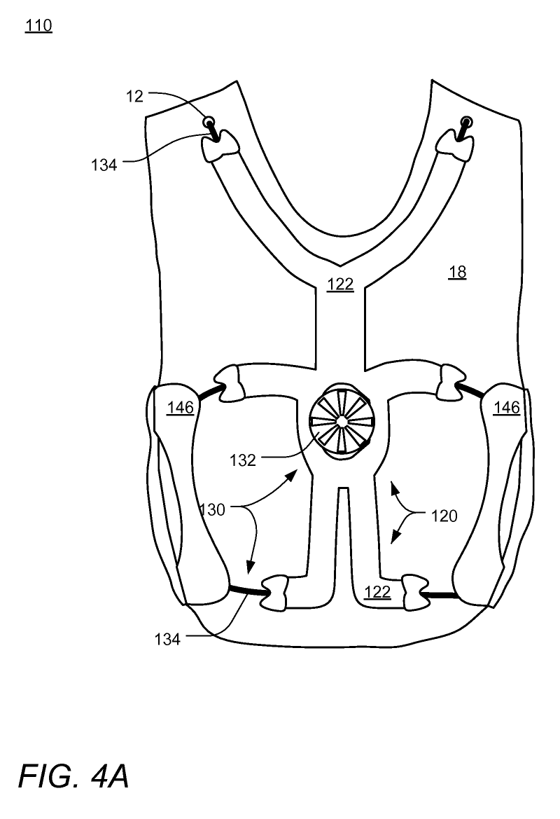

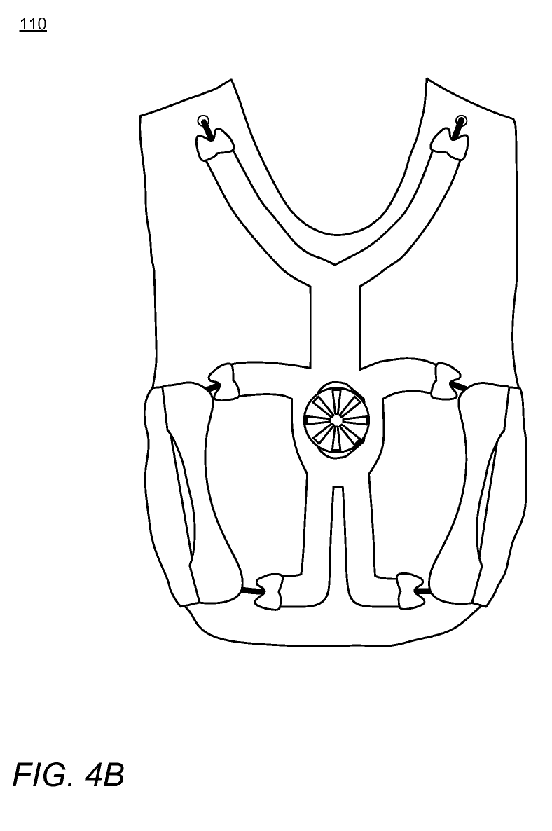

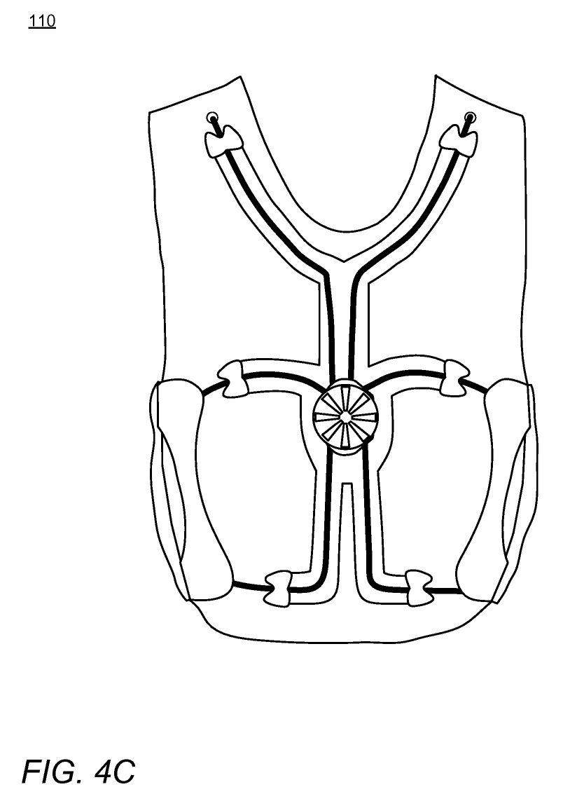

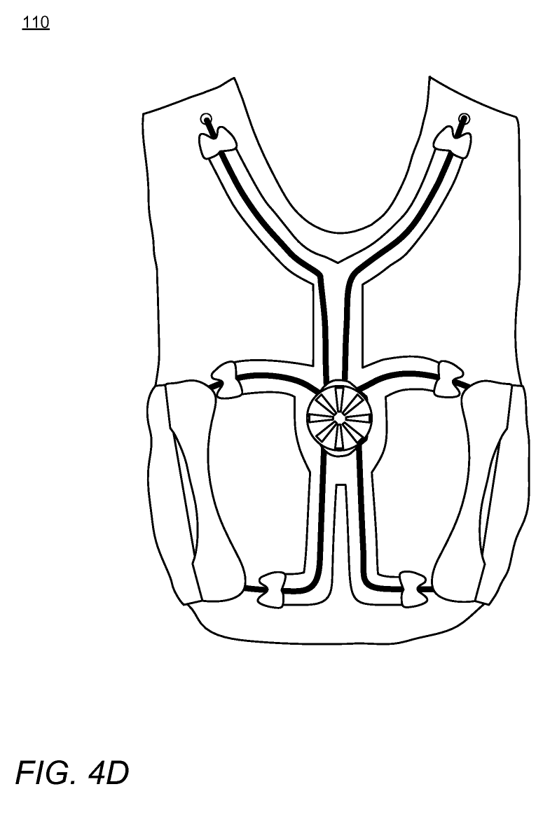

FIGS. 4A-4D are front views of an article of adjustably fitted protective apparel 110 according to a second embodiment of the present invention. As with the first embodiment of FIGS. 2A-2C, the article of protective apparel 110 comprises one or more protective panels 12, an inner cover (not shown), an outer cover 18, a channel distribution system 120 and an adjustable tensioning system 130. In this embodiment, the adjustable tensioning system 130 comprises a rotational tension adjuster 132 and a number of tensioning lines 134. First end portions of the tensioning lines 134 are spooled or otherwise gathered within the rotational tension adjuster 132. The tensioning lines 134 extend from the rotational tension adjuster 132 proximal the sternum area of the vest 110 and pass through distribution channels 122 formed in the front portion of the vest. Second end portions of the tensioning lines 134 are attached to anchor points 144 of the vest 110. As with the first embodiment, manipulation of the rotational tension adjuster 132 in one sense effects the withdrawing of portions of the tensioning lines 134 into the adjuster 132, thereby tightening of the vest 110. Manipulation of the rotational adjuster 132 in another sense effects the releasing of portions of the tensioning lines 134 from the adjuster 132, thereby loosening the vest 110.

The distribution channels 122 formed in the front portion of the vest 110 are made more apparent in FIGS. 4C and 4D by the visibility of the tensioning lines 134. Six tensioning lines are shown extending from the rotary tension adjuster in the illustrated embodiment of the vest 110 though other embodiments according to the present invention provide other numbers of tensioning lines 134 and other patterns of distribution channels 122. A respective shoulder tensioning line 134 extends from the adjuster 132 and toward each shoulder. A respective upper side torso tensioning line 134 extends from the adjuster 132 and toward each torso side passing just below the pectoral muscle. A respective lower side torso tensioning line 134 extends from the adjuster 132 and toward each torso side just above the hip. Each tensioning line 134 is optionally housed in a cable guide, rigid or flexible housing, or other jacketing to minimize binding between the line 134 and the vest 110.

Each tensioning line 134 is generally strong and capable of supporting tension, is flexible and is capable of being wound about a spool. Tensioning lines 134 each may comprise a mono-filament material or a multi-filament material and may be natural or synthetic fibers. Alternately the tensioning lines 134 may be formed of one or more metals and, for example, may comprise cables. Alternately the tensioning lines 134 may be formed from nylon.

Optionally, yoke portions of the vest 110 that extend from the pectoral area, passing over the collar bone area, and toward the shoulder blade area are constructed of extensible fabric that is elasticized to stretch and retract to avoid bunching or gathering of the material as the fit of the vest 110 is adjusted and as the wearer of the vest 110 moves about. Similarly, side portions of the vest 110 that extend from the frontal torso to the rear torso passing over the ribs under the arms are optionally constructed of extensible fabric. Thus, while protective front and back portions of the vest 10 may each comprise woven carbon fiber ballistic shield material that is not generally extensible, an adjustable and comfortable fit is facilitated by the rotary tension adjuster 132, multiple tensioning lines 134, and extensible yoke and side portions of the vest 110. A side portion of the vest 110 is shown in a loosened configuration in FIGS. 4A and 4C and in a tightened configuration in FIGS. 4B and 4D for comparison.

In FIGS. 4A-4D, the shoulder tensioning lines 134 that extend from the rotary tension adjuster 132 and toward the shoulders pass over the collar bone area, through channels 122 formed in the yoke portions of the vest 110, and are anchored to upper portions (not shown) of the back portion of the vest 110. Also shown, the upper and lower side torso tensioning lines 134 that extend from the rotary tension adjuster 132 and toward the side portion of the vest 110 are attached to the side portion of the vest 110 by way of a line spreader 146. The line spreader 146 maintains a distance between upper and lower side torso tensioning 134 lines, thereby preventing bunching or gathering of the vest 110 about the midriff as the wearer moves about.

In one variation of the vest 110 of FIGS. 4A-4D, the upper and lower side torso tensioning lines 134 are respective separate lines that are attached at opposite respective ends thereof to the line spreader 146 and rotary tension adjuster 132. In this variation, manipulation of the adjuster 132 effects adjustments of separate upper side torso and lower side torso lines.

In another variation of the vest 110 of FIGS. 4A-4D, the upper and lower side torso tensioning lines 134 are respective portions of a single line that extends from the rotation tension adjuster 132 sliding freely through the line spreader 146 and returning to the adjuster 132. The single line 134 is optionally anchored in a static sense to the rotary tension adjuster 132 at one end (for example, the lower torso line) and is spooled or otherwise gathered within the rotary tension adjuster 132 at the opposite end (for example, the upper torso line) such that manipulation of the adjuster 132 effects adjustments of the upper side torso portion the and lower side torso portion of the single line as the single line slides freely through the line spreader 146. Alternatively, the single line 134 is optionally spooled or otherwise gathered within the rotary tension adjuster 132 at respective upper torso and lower torso line ends that are each adjusted as the rotary tension adjuster 132 is manipulated.

In yet another variation (not shown) of the vest 110 of FIGS. 4A-4D, the upper and lower torso lines 134 pass through the line spreader 146 and through channels 122 formed in the side portions of the vest 110 and are attached or anchored to respective side margins of the back portion of the vest 110.

In still another variation (not shown) of the vest 110 of FIGS. 4A-4D, the left and right upper torso lines 134 are respective portions of a single line that fully encircles the wearer by extending from the rotary tension adjuster 132, passes through a left upper torso channel 122 formed in the front portion of the vest 110, passes through a left line spreader 146, passes through a channel 122 formed in the back portion of the vest 110, passes through a right line spreader 146, passes through a right upper torso channel 122 formed in the front portion of the vest 110, and returns to the adjuster 132. Similarly, in this variation, the left yoke line and right yoke line are similarly respective portions of a single line 134 that fully encircles the wearer by extending from the adjuster 132 and passes through a channel 122 formed in the upper back portion of the vest 110 and returns to the adjuster 132.

Although not shown, it will be understood that the vest 110 may be donned under a shirt. An advantage of the placement of the rotary tension adjuster 132, disposed along the axis defined by the vertical line of buttons of the shirt, is apparent as the adjuster 132 is easily exposed for manipulation by reaching between buttons or by releasing a minimum number of buttons of the shirt. Manipulation of the adjuster 132 is thereby possible without removing the shirt and without a need for un-tucking the shirt (when the bottom of the shirt is tucked into pants). Thus the vest 110 can be worn discreetly and readily adjusted conveniently.

As illustrated, the rotary tension adjuster 132 may be of the type described in U.S. patent application Ser. No. 10/129,900. However, other rotary tension adjusters 32 or cranks may also be used, including those of the other incorporated references, such as the coplanar cranks disclosed in U.S. patent application Ser. No. 11/276,357.

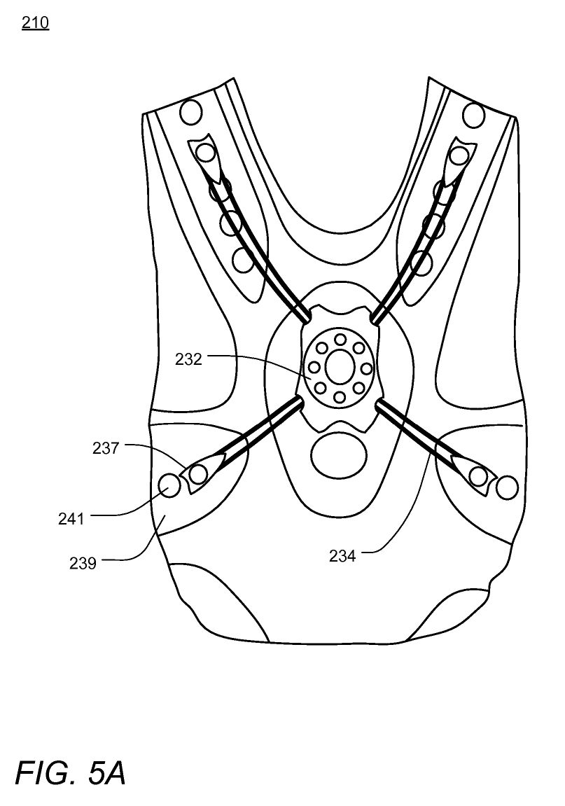

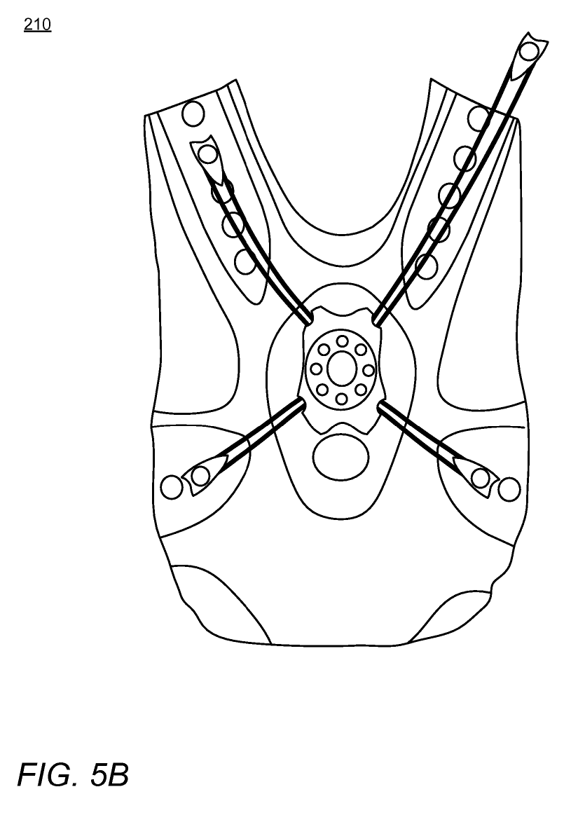

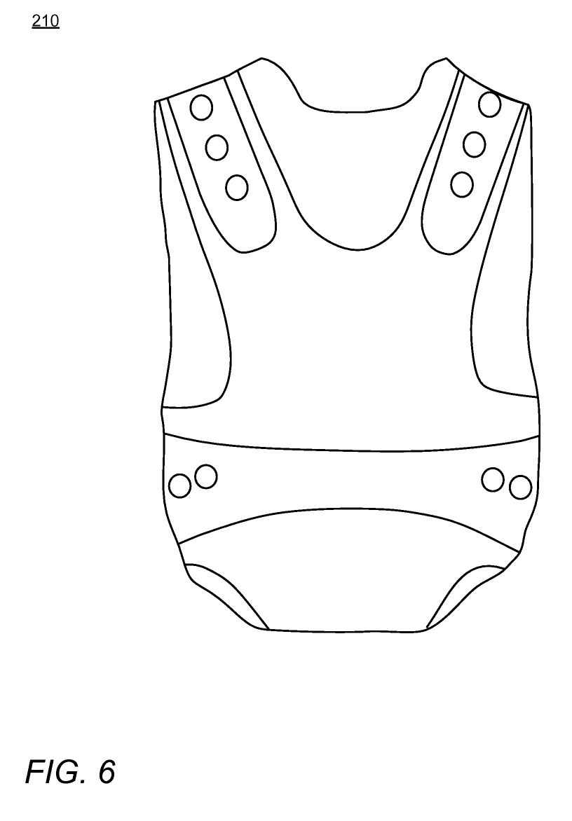

FIGS. 5A and 5B are front views of an article of adjustably fitted protective apparel 210 according to a third embodiment of the present invention, and FIG. 6 is a front view of the article of adjustably fitted protective apparel 210 of FIG. 5A. In this embodiment, four tension loops 234 extend from a rotary tension adjuster 232 centrally located at the sternum area of the front of the vest. Each loop 234 extends from the rotary tension adjuster 232 and through a respective clip 237. The clips 237 are each removably attached to holed bands 239 attached to the vest 210. Respective left and right yoke bands 239 extend from the front pectoral areas to the back shoulder blade areas of the vest 210. As shown in FIG. 6, a single waist band 239 passes across the lower portion of the back of the vest 210 and has respective opposite ends that extend beneath the arms along the ribs toward the adjuster 232. Each band 239 provides a number of holes 241 for receiving a clip 237 so that positioning each clip 237 serves as a macro-adjustment or coarse adjustment of the fit of the vest 210 on a wearer. Manipulation of the rotary tension adjuster 232 serves as a micro-adjustment or fine adjustment of the fit of the vest 210 on the wearer. Manipulation of the rotary tension adjuster 232 also serves as a convenient adjustment of the fit of the vest 210 without more labor and intensive movement of the clips 239.

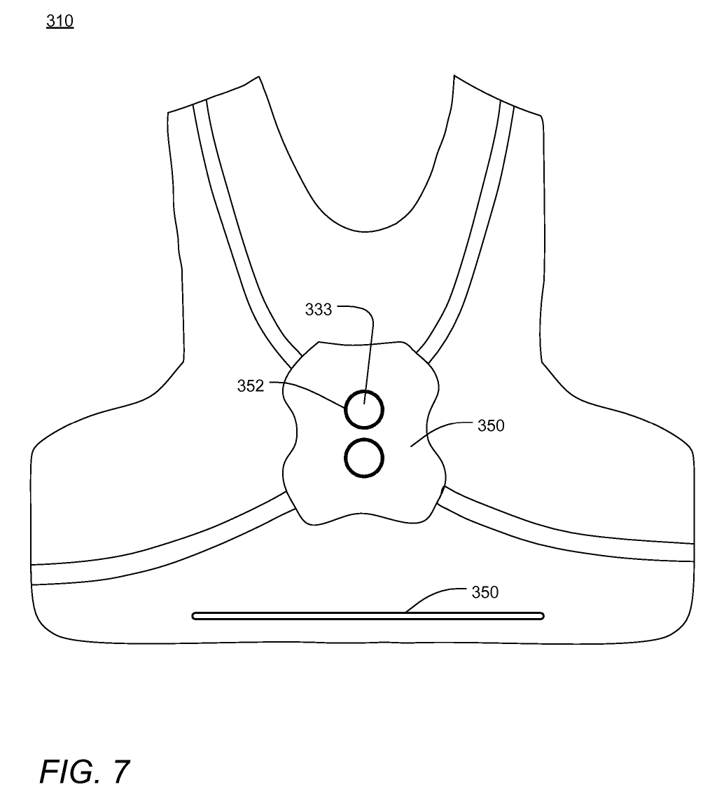

FIG. 7 is a front view of an article of adjustably fitted protective apparel 310 according to a fourth embodiment of the present invention. In this embodiment, a receiving pocket 250 is formed in the article for receiving a tension adjuster. One or more apertures are defined in the pocket for extension there through of control elements of a tension adjuster so that the tension adjuster can be manipulated without removal from the pocket. Furthermore, in this embodiment, yoke portions of the article comprise crossed belts, for example woven nylon belts, that provide strength and that are covered by fabric portions for comfort.

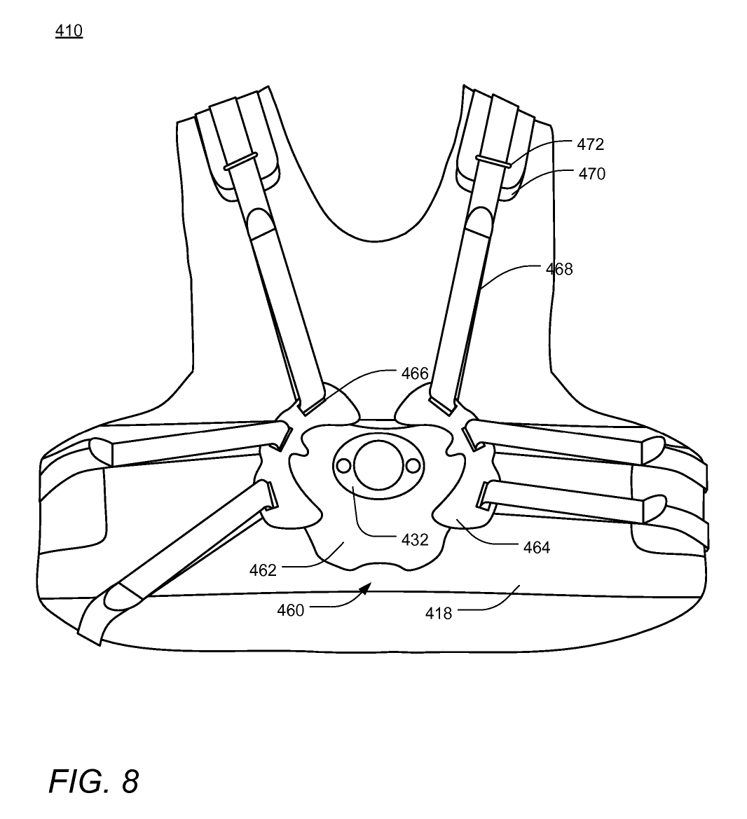

FIG. 8 is a front view of an article of adjustably fitted protective apparel 410 according to a fifth embodiment of the present invention. In this embodiment, a rotary tension adjuster assembly 460 is positionable upon a receiving area of the front of the vest 410. The rotary tension adjuster assembly 460 includes a rotary tension adjuster 432, a positionable base plate 462 constructed of see-through plastic, and a pair of joiners 464. The positionable base plate 462 has attachment portions for removably attaching the base plate 462 to the receiving area 460 of the front of the vest 410. In the illustrated embodiment, the receiving area 460 of the vest 410 comprises loop fabric and the attachment portions of the positionable base plate 462 comprise hook fabric for releasably engaging the loop fabric of the receiving area 460 in hook-and-loop engagement. Each joiner 464 links a tension line (not shown) extending from the rotary tension adjuster 432 to multiple tension belts 436. In the illustration embodiment, each joiner 464 links a tension line to three tension belts 436. Manipulation of the rotary tension adjuster 432 effects adjustment of the tension belts 436 by way of the joiner 464 linking the belts 436 to a tension line that is directly adjusted by the manipulation. Each tension belt 436 passes through a slot 466 formed in one or the other of the joiners 464 and releasably and adjustably attaches to itself by way of hook-and-loop fabric areas 468 in hook-and-loop engagement. Each tension belt 464 is thereby individually adjustable by way of adjustable self-engagement serving as a macro-adjustment or coarse adjustment of the fit of the vest 410 on a wearer. Manipulation of the rotary tension adjuster 432 serves as a micro-adjustment or fine adjustment of the fit of the vest 410 on the wearer. Further control may be provided using additional hook-and-loop panels 470, placed on the shoulders, hips or the like, and guide rings 472.

The rotary tension adjuster 432 in FIG. 8 optionally comprises a rotary device for tensioning and releasably locking a spooled cable. For example, U.S. Pat. No. 5,042,177 to Schoch, titled “Rotary Closure for a Sports Shoe, Especially a Ski Shoe,” which patent is hereby incorporated herein by reference, describes such a rotary device. This rotary tension adjuster or crank 432 may also be used in other embodiments of the invention disclosed herein, and the cranks 32, 132, 232, 332, 432, 632 of those embodiments may be used in the embodiment of FIG. 8.

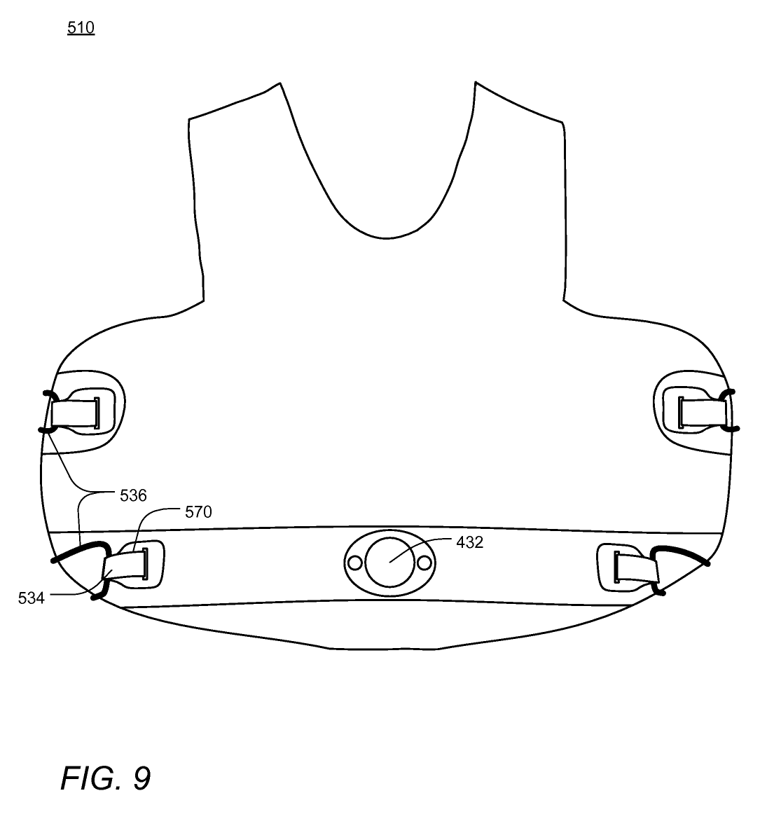

FIG. 9 is a front view of an article of adjustably fitted protective apparel 510 according to a sixth embodiment of the present invention. In this embodiment, a rotary tension adjuster 432 is positioned on the front of the vest 510 and a tension line 534 extends from each side (left and right) of the adjuster to a respective serpentine side cord 536. The serpentine side cords 536 adjustably attach front and rear side portions of the vest 510. The serpentine side cords 536 pass freely through loops 570 attached to the front and rear side portions of the vest 510 in an alternating serpentine fashion. Manipulation of the rotary tension adjuster 432 effects adjustment of the tension lines 534 which effect adjustment of the serpentine side cords 536. The loops 570 of the rear side portion of the vest 510 may be held spaced apart by a rigid arcuate spreader bar (not shown) to prevent bunching, gathering and migration of the sides of the vest 510.

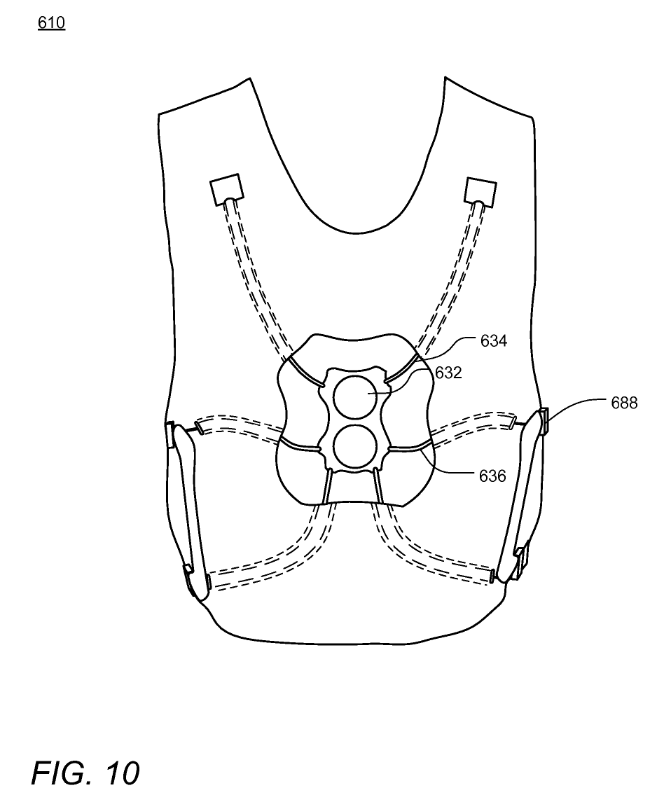

FIG. 10 is a front view of an article of adjustably fitted protective apparel 610 according to a seventh embodiment of the present invention. In this embodiment, which has many similarities to that of FIGS. 4A-4D, provides a pair of rotary tension adjusters 632 to facilitate adjustments of shoulder tensioning lines 634 separately from adjustments of upper and lower side torso tensioning lines 636. Quick-release buckles 680 are provided in this embodiment releasably connecting line spreaders 646 to lower side margins of the back of the vest 610. The quick-release buckles 680 allow the vest 610 to be donned and removed quickly and conveniently as desired or as needed in an emergency situation. When released, the quick-release buckles 680 also allow for the vest 610 to be worn hanging from the shoulders by the yoke portions of the vest 610 when the wearer is comfortable that a threat does not exist and desires comfort, such as when enjoying a meal.

As stated with regard to one or more of the previous embodiments of the present invention, it will be appreciated from the foregoing that the control for the rotary tension adjuster is easily accessed for manipulation such as, for example, by reaching between buttons of a shirt or by releasing a minimum number of buttons of the shirt for rotation of the control dial. Thus, a vest can be worn discreetly and readily adjusted conveniently while being worn. While the embodiments shown in the figures and described in detail herein relate particularly to upper torso protective ballistic vests, the invention is applicable to protective apparel in general. For example, the invention is applicable to personal floatation devices, life vests, life jackets, water ski vests, and wake board vests. Furthermore, the invention is applicable to body armor for military and police personnel, as well as to any protective apparel worn by citizens or protective gear worn by athletes. Based on the foregoing description, it will be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention. Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements, the present invention being limited only by the claims appended hereto and the equivalents thereof.

What is claimed is:

1. An article of adjustably fitted protective apparel, comprising:
(a) a vest; and
(b) an adjustable tensioning system for selective tensioning of the vest, comprising,
(i) a rotary tension adjuster disposed on the vest and including a housing and a first spool disposed within the housing, and
(ii) a first tensioning line extending from the housing and coupled directly or indirectly to the vest, a portion of the first tensioning line configured to be wound upon the first spool during manipulation of the rotary tension adjuster in a first rotational direction thereof, winding of the portion of the first tensioning line on the first spool drawing first portions of the vest together for providing a snug fit of the first portions of the vest when worn;
(c) wherein the adjustable tensioning system further comprises a distribution channel system through which the tensioning line extends, a first distribution channel of the distribution channel system sheathing the first tensioning line.

2. The article of protective apparel of claim 1, wherein the first distribution channel comprises an elongate section that sheathes the first tensioning line and an outrigger section that extends from the elongate section to provide stability and support to the elongate section during tensioning of the first tensioning line.

3. The article of protective apparel of claim 1, wherein the first distribution channel comprises a layer of material that is coupled to a surface of the vest in overlapping relation to the first tensioning line.

4. The article of protective apparel of claim 3, wherein the first distribution channel further comprises a tubular member through which the first tensioning line extends, the layer of material overlapping the tubular member through which the first tensioning line extends.

5. The article of protective apparel of claim 1, wherein the distribution channel system includes a C-shaped section proximate a side portion of the vest.

6. The article of protective apparel of claim 1, wherein the distribution channel system includes a C-shaped section proximate a shoulder portion of the vest.

7. The article of protective apparel of claim 1, wherein the first tensioning line fully encircles a wearer of the vest by extending from the rotary tension adjuster, passing through a left upper torso distribution channel formed in a front portion of the vest, passing through a left line spreader, passing through a distribution channel formed in a back portion of the vest, passing through a right line spreader, passing through a right upper torso distribution channel formed in another front portion of the vest, and returning to the rotary tension adjuster.

8. An article of adjustably fitted protective apparel, comprising:
(a) a vest; and
(b) an adjustable tensioning system for selective tensioning of the vest, comprising,
(i) a rotary tension adjuster disposed on the vest and including a housing and a first spool disposed within the housing, and
(ii) a first tensioning line extending from the housing and coupled directly or indirectly to the vest, a portion of the first tensioning line configured to be wound upon the first spool during manipulation of the rotary tension adjuster in a first rotational direction thereof, winding of the portion of the first tensioning line on the first spool drawing first portions of the vest together for providing a snug fit of the first portions of the vest when worn;
(c) wherein a second tensioning line extends from the housing and is coupled directly or indirectly to the vest, a portion of the second tensioning line configured to be wound upon the first spool during manipulation of the rotary tension adjuster in the first rotational direction thereof, winding of the portion of the second tensioning line on the first spool also drawing the first portions of the vest together for providing a snug fit of the first portions of the vest when worn, and wherein the first tensioning line comprises an upper side torso tensioning line that extends from the rotary tension adjuster toward a side portion of the vest, and wherein the second tensioning line comprises a lower side torso tensioning line that extends from the rotary tension adjuster toward the side portion below and in spaced relation to the upper side torso tensioning line.

9. The article of protective apparel of claim 8, wherein both the upper side torso tensioning line and the lower side torso tensioning line are coupled to the side portion of the vest by way of a line spreader.

10. The article of protective apparel of claim 9, wherein the line spreader is coupled to a back portion of the vest by a quick-release buckle.

11. The article of protective apparel of claim 9, wherein the line spreader comprises a rigid, elongate member that maintains a fixed distance between opposed ends thereof, and wherein the upper and lower side torso tensioning lines are coupled to the line spreader at the respective opposed ends thereof, whereby bunching or gathering of the vest about the midriff of the wearer is prevented during tensioning of the side portion.

12. The article of protective apparel of claim 11, wherein the upper and lower side torso tensioning lines comprise separate lines that are coupled at opposite respective ends thereof to the line spreader and to the rotary tensioner adjuster.

13. The article of protective apparel of claim 11, wherein the upper and lower torso lines pass through the line spreader and through distribution channels formed in the side portion of the vest for coupling of the lines to a back portion of the vest.

14. The article of protective apparel of claim 8, wherein the upper and lower side torso tensioning lines are respective portions of a single line.

15. The article of protective apparel of claim 14, wherein the single line forming the upper and lower side torso tensioning lines slides freely through a channel defined by the line spreader.

16. The article of protective apparel of claim 15, wherein a first end of the single line is fixedly attached to the rotary tension adjuster and wherein a second end of the single line is spooled within the rotary tension adjuster.

17. The article of protective apparel of claim 15, wherein opposite ends of the single line both are spooled within the rotary tension adjuster.

18. A method for adjustably tensioning an article of protective apparel, comprising the step of manipulating a rotary tension adjuster in a first rotational direction thereof to draw together first sections of a vest.

19. The method of claim 18, further comprising the step of manipulating the rotary tension adjuster in a second rotational direction thereof opposite to the first rotational direction thereof to draw together second sections of the vest.

20. The method of claim 19, wherein each step of manipulating the rotary tension adjuster comprises manually rotating a dial of the rotary tension adjuster.
